(19) United States
(12) Patent Application Publication
(10) Pub. No.: US 2020/0020438 A1
(43) Pub. Date: Jan. 16, 2020

(54) Computer Search Engine for Healthcare Outcome Efficiency

(71) Applicants: Jack McCallum, Benbrook, TX (US); Scott Roloff, Arlington, TX (US); William McCallum, Forth Worth, TX (US); Ken Grifno, The Colony, TX (US)

(72) Inventors: Jack McCallum, Benbrook, TX (US); Scott Roloff, Arlington, TX (US); William McCallum, Forth Worth, TX (US); Ken Grifno, The Colony, TX (US)

(73) Assignee: Integer Health Technologies, LLC, Forth Worth, TX (US)

(21) Appl. No.: 16/031,559

(22) Filed: Jul. 10, 2018

(51) Int. Cl. G16H 40/20; G16H 50/30; G06F 17/30; G06Q 10/10

(52) U.S. Cl. CPC G16H 40/20; G16H 50/30; G06N 3/08; G06F 17/30619; G06Q 10/10; G06F 17/30867

(57) ABSTRACT

A computer search engine for medical and/or pharmacy claims (in combination with employer human resource records or on a stand-alone basis) that ranks healthcare providers and/or intervention strategies by root diagnosis based upon their overall average outcome efficiency. Outcome efficiency is the adjusted cost per day to keep a patient functional (or in the case of an employer, keep an employee at work), so the lower the outcome efficiency the better. The search engine uses drop-down menus and/or similar techniques that require the user to select a root diagnosis on which to search, as well as other variables (e.g. provider category, geographic proximity, in-network versus in or out of network, etc.), turning an open-ended question, e.g. “Which doctor should I go to for back pain?” to a closed-ended one “Which surgeons in my network within 25 miles have the best outcome efficiencies for back surgery?”

### TECHNICAL FIELD

The invention is a computer search engine that ranks healthcare providers and intervention strategies by root diagnosis based upon quantified outcomes—the cost per day to keep an individual functional (i.e. outcome efficiency), so the lower the cost the better.

### BACKGROUND

A need exists to search for healthcare providers and intervention strategies based on measurable outcomes.

To date, various means exist to measure the “inputs” to the healthcare system, such as the processes that a provider follows when treating a patient or whether the patient liked or disliked the provider.

There is an unsatisfied need, however, to quantify the “output,” whether the patient actually got better, and how much that cost.

### SUMMARY

This computer search engine searches medical and pharmacy claims data (by themselves or in combination with employer human resource records) to determine the average outcome efficiency of a healthcare provider and/or intervention strategy when treating patients with a particular root diagnosis.

Outcome efficiency is the cost per day to keep a patient functional, so the lower the cost the better.

This invention therefore measures healthcare output.

### BRIEF DESCRIPTION OF THE DRAWINGS

A complete understanding of this invention may be obtained by reference to the accompanying drawings in conjunction with the following detailed description.

FIG. 1 depicts the computer search engine's flow when searching medical and pharmacy claims only, with a cut-out for sorting and tasking the claims by both provider and intervention strategy, which is shown in FIG. 2, and a cut-out for determining a patient's functional days, which is shown in FIG. 3.

FIG. 2 depicts the computer search engine's flow for sorting and tasking the claims by both provider and intervention strategy.

FIG. 3 depicts the computer search engine's flow for determining a patient's functional days when searching medical and pharmacy claims only.

FIG. 4 depicts the computer search engine's flow when searching medical and pharmacy claims in combination with employer human resource records, with a cut-out for sorting and tasking the claims by both provider and intervention strategy, which is shown in FIG. 2, and a cut-out for determining an employee's functional days, which is shown in FIG. 5.

FIG. 5 depicts the computer search engine's flow for determining an employee's functional days when searching medical and pharmacy claims in combination with employer human resource records.

FIG. 6 depicts the computer search engine's flow when predicting the outcome efficiency with respect to a particular patient or employee when going to a specific provider, or undergoing a specific intervention strategy, for treatment of a root diagnosis, and then the invention's neural network learning feature.

### DETAILED DESCRIPTION

Various objects, features, aspects and advantages will become apparent from the following detailed description along with the accompanying drawings. The principles are described with specificity. This description and the drawings, however, are not intended to limit the scope of the principles disclosed herein. These principles might also be embodied in other ways and include different steps or combinations of steps similar to the ones described.

This invention is a computer search engine for medical and pharmacy claims data to determine the average outcome efficiency of a healthcare provider and/or intervention strategy when treating patients with a particular root diagnosis.

This search engine can be deployed only on claims data, or on claims data in combination with employer human resource records. When the search engine searches both claims and human resource records, the results reported concern only the employees of the employer, not any other individuals covered under the employer's health plan.

In addition, the claims data and human resource records from different plans and/or employers can be pooled to increase the data set and provide more robust results.

The search engine displays different results depending on the user.

In each case, the search engine uses drop-down menus and/or similar techniques that require the user to select a root diagnosis on which to search, as well as other variables (e.g. provider category, in-network versus in or out of network, geographic proximity, etc.), turning an open-ended question, e.g. “Which doctor should I go to for back pain?” to a closed-ended one “Which surgeons in my network within 25 miles have the best outcome efficiencies for back surgery?”

If the user is a health plan, provider network, employer, or other administrative user, the search engine displays the overall average outcome efficiency by root diagnosis of each healthcare provider treating patients with that diagnosis, and each available intervention strategy for that diagnosis, including configurations and subsets in various dashboards and reports.

If the user is a primary care physician (PCP) or other healthcare provider that needs to make a patient referral or choose from several available intervention strategies, the search engine displays the overall average outcome efficiencies by root diagnosis of the healthcare specialists, surgeons and institutions treating patients with that diagnosis that have overall average outcome efficiencies better than or equal to a designated threshold and/or the overall average outcome efficiencies of the possible interventions. For example, if the search engine is set to display only specialists, surgeons and institutions whose overall average outcome efficiency is better than the average for that category, the search engine will only display those providers whose overall average outcome efficiency is below that average (the outcome efficiency is the adjusted claims cost per day to keep a patient functional, or an employee at work, so the lower the cost the better).

If the user is an individual seeking treatment, the search engine displays the overall average outcome efficiencies by root diagnosis of all healthcare providers (e.g. PCPs, specialists, surgeons and institutions) treating patients with that diagnosis that have overall average outcome efficiencies better than or equal to the designated threshold. In this scenario the search engine could also display the overall average outcome efficiencies of the possible intervention strategies, but may not do so because individuals without a medical background may not have the experience required to interpret the results.

In addition to (or in replacement of) the overall average outcome efficiencies displayed to PCPs and other healthcare providers needing to make referrals,, or choose from several intervention strategies, as well as individuals seeking treatment, the search engine can display the predicted outcome efficiency for a provider when treating a particular patient, or for a particular intervention strategy, by taking the overall average outcome efficiency for the provider or strategy and multiplying it by the individual's risk score, and if the individual is an employee, then multiplying the resulting product by the employee's job factor too (before or after the risk score and job factor are modified through the regression analysis and neural network loop described below).

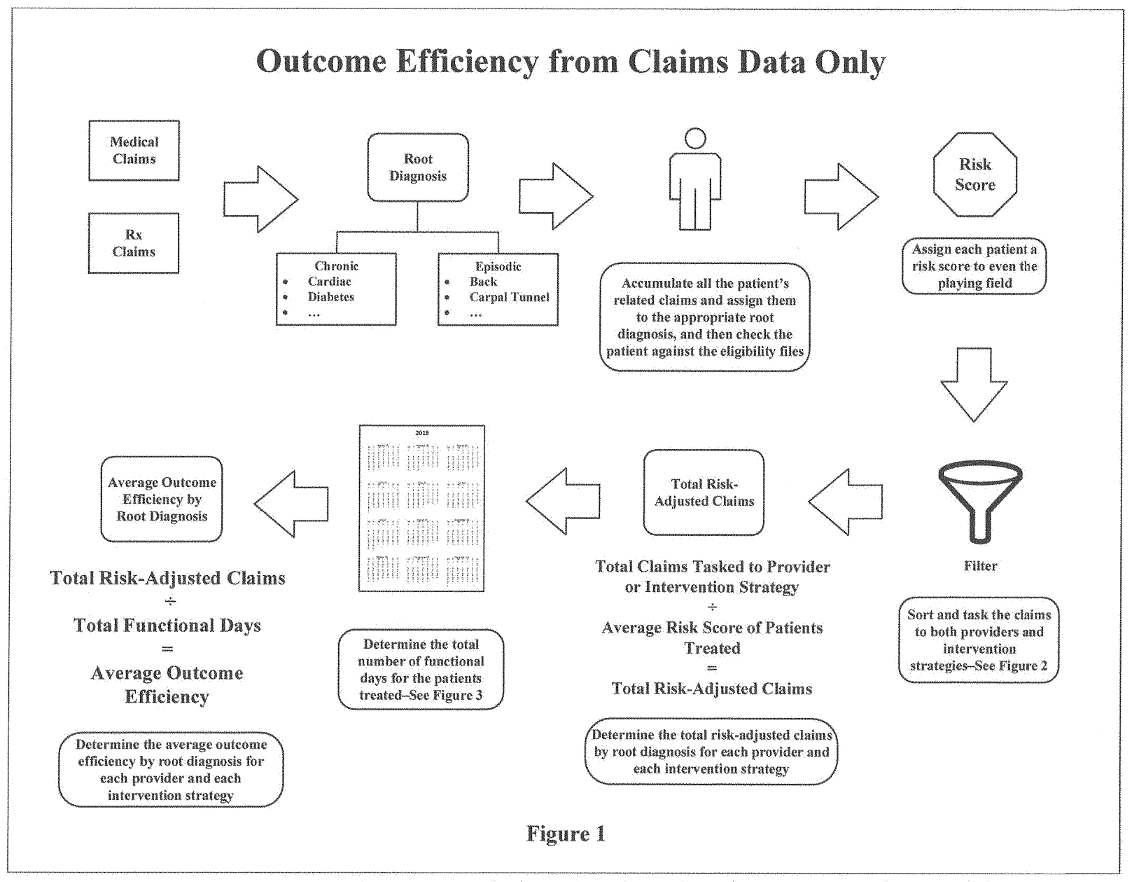

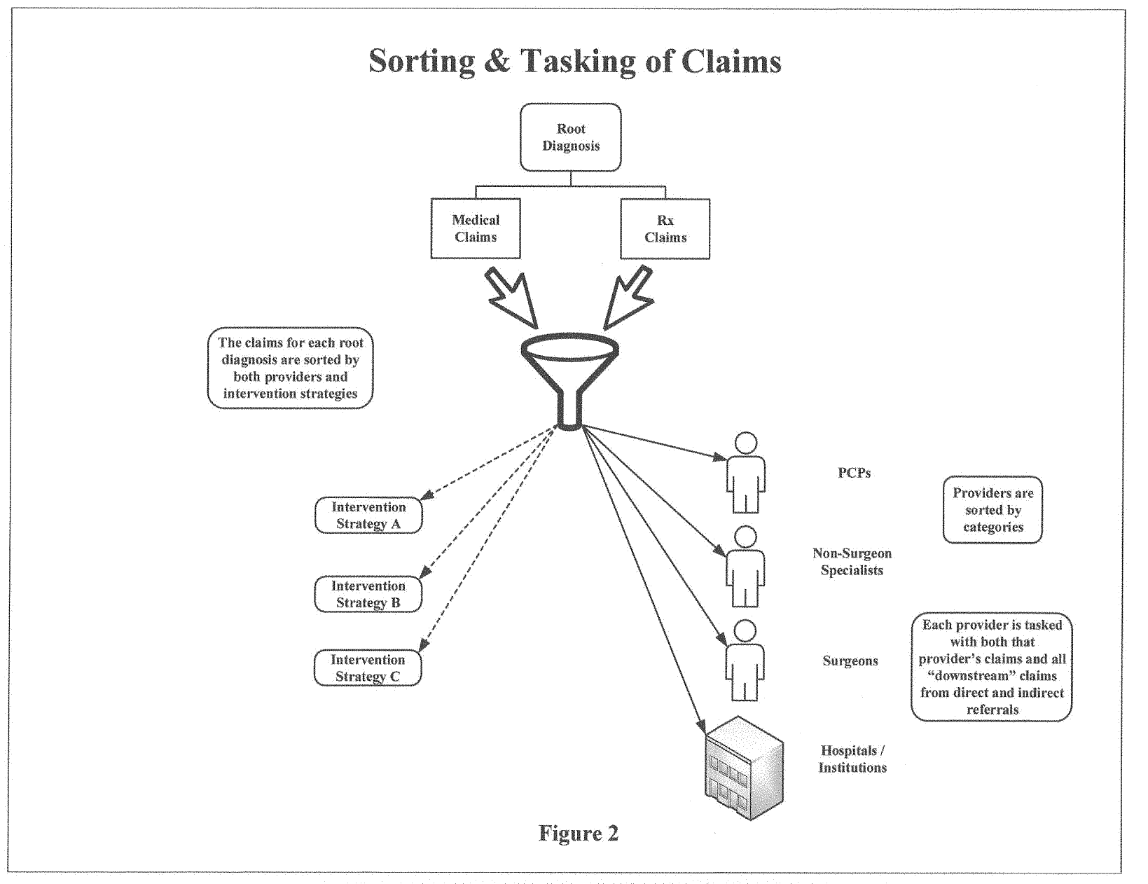

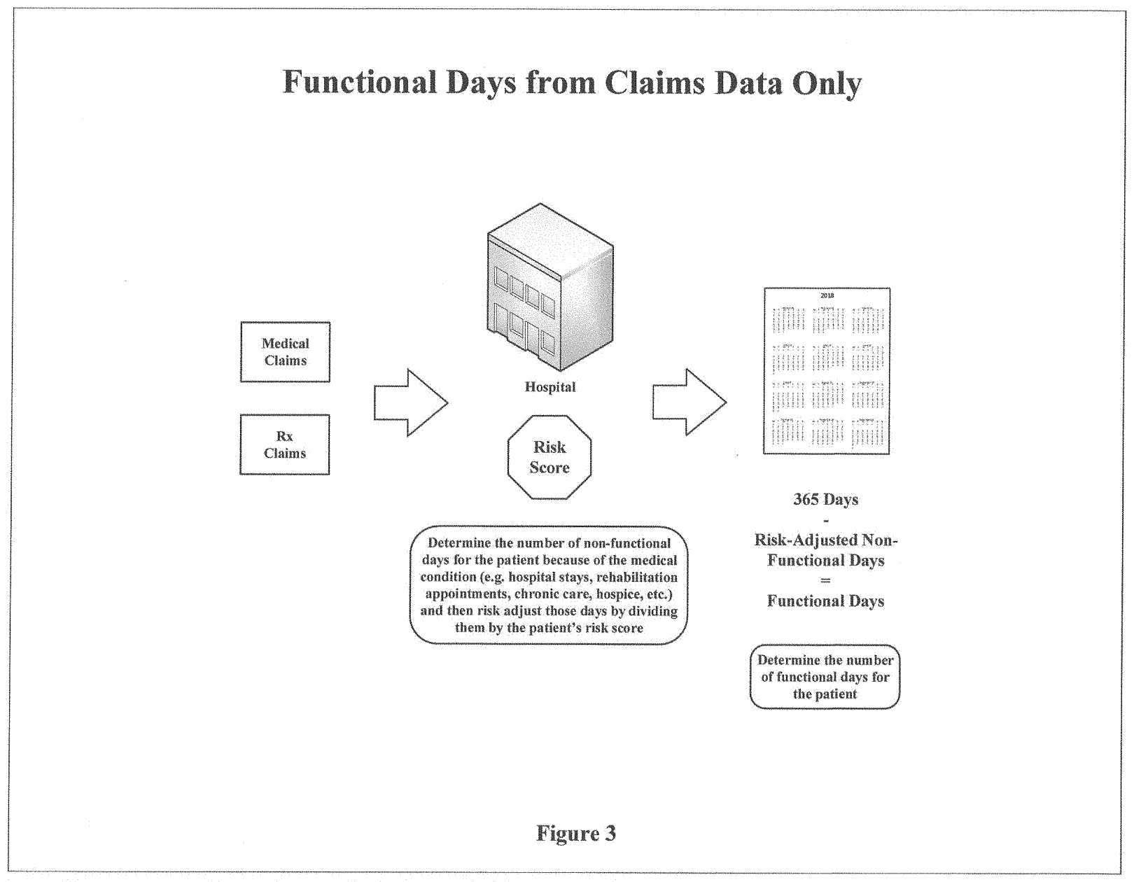

FIG. 1 depicts the computer search engine's flow when searching medical and pharmacy claims only, with a cut-out for sorting and tasking the claims by both provider and intervention strategy, which is shown in FIG. 2, and a cut-out for determining a patient's functional days, which is shown in FIG. 3. The search engine works best when searching both medical and pharmacy claims, but can be used on just medical claims without pharmacy claims (and under certain circumstances, on just pharmacy claims without medical claims). This detailed description and the drawings assume deployment on both.

First, the search engine organizes the medical and pharmacy claims in tables with various headers that enable filtering, grouping and matching. The search engine then sifts through all the claims and identifies the root diagnosis for each patient's claims (or root diagnoses, if more than one). A “root diagnosis” is the patient's main problem from which all related claims emanate. There are two main categories of root diagnoses: chronic conditions (e.g. cardiac problems, diabetes, etc.) and episodic conditions (e.g. back pain, carpal tunnel syndrome, etc.). Under each main category there are many root diagnoses.

Next, the search engine accumulates all the medical and pharmacy claims attributable to a patient's root diagnosis.

The search engine then groups the claims by measurement period, such as an annual period (e.g. calendar years or rolling twelve-month periods). Under certain circumstances the search engine could use something other than an annual period. For example, the search engine could determine the average number of days for an episodic root diagnosis and use that average as the measurement period. The default measurement period, and the one assumed for the balance of this description and the drawings, is an annual period.

After that the search engine organizes the eligibility files for any applicable health plan or other program in tables with various headers that enable filtering, grouping and matching, and determines whether each patient participated in that plan or program for the entirety of each measurement period. The search engine may discard from the analysis patients to the extent that they only participate for part of a period.

The search engine then analyzes the claims to assign each patient a risk score denoting the patient's overall health based on the patient's age, gender, diagnoses, and in some cases, drug prescriptions. Various risk scoring systems exist. For example, the Hierarchical Condition Category (HCC) system is used by Medicare, while the Chronic Illness and Disability Payment System (CDPS) is used by many Medicaid programs. The search engine normalizes the risk scores so that individuals of average health may receive a risk score of 1.000, individuals healthier than average a score below 1.000 (the lower the score, the healthier), and individuals sicker than average a score above 1.000 (the higher the score, the sicker). To further normalize the results, the search engine may assign a patient with a score of 1.000 or below a score of 1.000, while using the actual scores for individuals with scores above 1.000.

Next the search engine organizes the healthcare providers and intervention strategies (including employee assistance programs, such as for alcohol abuse and depression) in tables with various headers that enable filtering, grouping and matching.

Each provider is then tasked with both: (1) that provider's claims, and (2) all “downstream” claims from direct and indirect referrals of the patient made by that provider to other providers. Note that if all the claims of all the providers were added together that this would result in double, triple counting, etc., although for ranking purposes it doesn't matter. For example, if a PCP treated a patient and then referred that patient to a specialist, that PCP's claims would be not only the claims related to the PCP's treatment, but all the specialist's claims too. In addition, the specialist's claims would be attributed to the specialist, along with any downstream costs of further referrals (which would also be included in the PCP's costs). This attribution permits evaluation of referral patterns, which is essential when determining the outcome efficiency of a provider.

The search engine then sorts the providers into categories (i.e. you cannot compare a PCP to a surgeon). For example, the search engine might sort the providers into four categories: PCPs (including physician assistants'and nurse practitioners), non-surgeon specialists, surgeons, and institutions (e.g. hospitals, out-patient centers, etc.).

Similarly, the search engine sorts the claims by intervention strategy, tasking each strategy with both the direct claims of that intervention strategy and the indirect “downstream” claims stemming from it. Note that the search engine sorts the claims by both providers (and can assign the same claim to more than one provider) and by intervention strategy.

For each provider and/or intervention strategy the search engine then determines the risk-adjusted claims cost for each patient, by root diagnosis, for each measurement period, by taking the aggregate claims for that patient and diagnosis tasked to that provider or intervention strategy for that period, and dividing by the patient's risk score for that period. This risk adjustment gives credit for caring for sicker patients, who you would expect to cost more.

To obtain the total risk-adjusted claims for each root diagnosis for each provider and/or intervention strategy for each measurement period the search engine aggregates all the claims costs for that diagnosis tasked to the provider or intervention strategy for that period and divides that total by the average risk score for that period of the patients with that root diagnosis that the provider treated or who underwent that intervention.

Next, the search engine determines the average outcome efficiency for each provider or intervention strategy for each measurement period for that root diagnosis by taking the total risk-adjusted claims for that diagnosis tasked to that provider or intervention strategy and dividing by the total functional days in the measurement period of the patients that the provider treated for that diagnosis or who underwent that intervention.

As discussed above, the default measurement period is an annual period, so the total number of possible functional days is 365.

To determine a patient's functional days, the search engine begins by sifting through the claims and identifying each patient's non-functional days related to a root diagnosis. A “non-functional day” is a day when the patient was not functioning according to the patient's normal activities because of that diagnosis. Such non-functional days could include days involving: in-patient hospital admissions, out-patient hospital admissions, in-patient rehabilitation, out-patient rehabilitation, in-patient chronic care, out-patient chronic care, and hospice.

After that the search engine risk adjusts the patient's non-functional days by dividing them by the patient's risk score.

A patient's functional days are the total possible functional days in the measurement period—365—less the adjusted number of non-functional days.

The outcome efficiency for a provider and/or intervention strategy for a measurement period with respect to a patient with a particular root diagnosis is therefore the risk-adjusted claims cost tasked to that provider or intervention strategy with respect to that patient divided by that patient's functional days. For example, take a patient with diabetes, a chronic root diagnosis. The PCP treating that patient is tasked with $1,500 of claims costs during the year when treating that patient for that root diagnosis. The patient's risk score is 1.200, so the patient's risk-adjusted claims cost is $1,250 ($1,500÷1.200=$1,250). If the patient has 20 non-functional days because of the diabetes, the risk-adjusted number of non-functional days would be 17 (20÷1.200=17). The patient's functional days would therefore be 348 (365−17=348). Accordingly, the PCP's outcome efficiency for treating this patient for diabetes would be $3.59 ($1,250÷348=$3.59). This is the cost per day to keep this patient functional.

The average outcome efficiency for a provider and/or intervention strategy for a root diagnosis over a year would be the total claims cost of all the patients with that diagnosis tasked to the provider or intervention strategy during that period, divided by the average risk score of those patients during that period, and then divided by their total functional days. When determining this average, outliers may be excluded, e.g. a patient on which the outcome efficiency is more than three standard deviations from the mean. In addition, providers and intervention strategies with less than a minimum number of patients who were treated for that root diagnosis, or who underwent that intervention strategy, may also be excluded. For example, assume that the PCP from the previous example treated ten patients during the year for diabetes and that the total claims costs tasked to the PCP was $19,500. The average risk score of the ten patients was 1.300, so the PCP's total risk-adjusted claims cost was $15,000 ($19,500÷1.300=$15,000). If the ten patients had a total of 300 non-functional days because of their diabetes, the risk-adjusted number of non-functional days would be 231 (300÷1.300=231). The total functional days would therefore be 3,419 ((10×365)−231=3,419). Accordingly, the PCP's average outcome efficiency for treating these ten patients for diabetes would be $4.39 ($15,000÷3,419=$4.39).

When ranking the providers and/or intervention strategies by root diagnosis, the search engine may use the overall average of the annual outcome efficiencies for that diagnosis of each provider or strategy over a designated period using a simple average, weighted average or other means.

The search engine can also act on medical and pharmacy claims in combination with employer human resource records. In this scenario, outcome efficiency is determined on only employee data because there is no non-employee human resource data to match against the non-employees' medical and pharmacy claims. The outcome efficiency rankings derived from the employee data, however, can be used by employees and non-employees alike (e.g. spouses, dependent children, etc.) to identify the best providers and intervention strategies for what they need, as well as by employers when directing care in connection with their workers' compensation programs.

Alternatively, when using human resource records the search engine could take a bifurcated approach, determining the outcome efficiencies with respect to the employees using those human resource records while determining the outcome efficiencies with respect to the non-employees from only the claims data as discussed above.

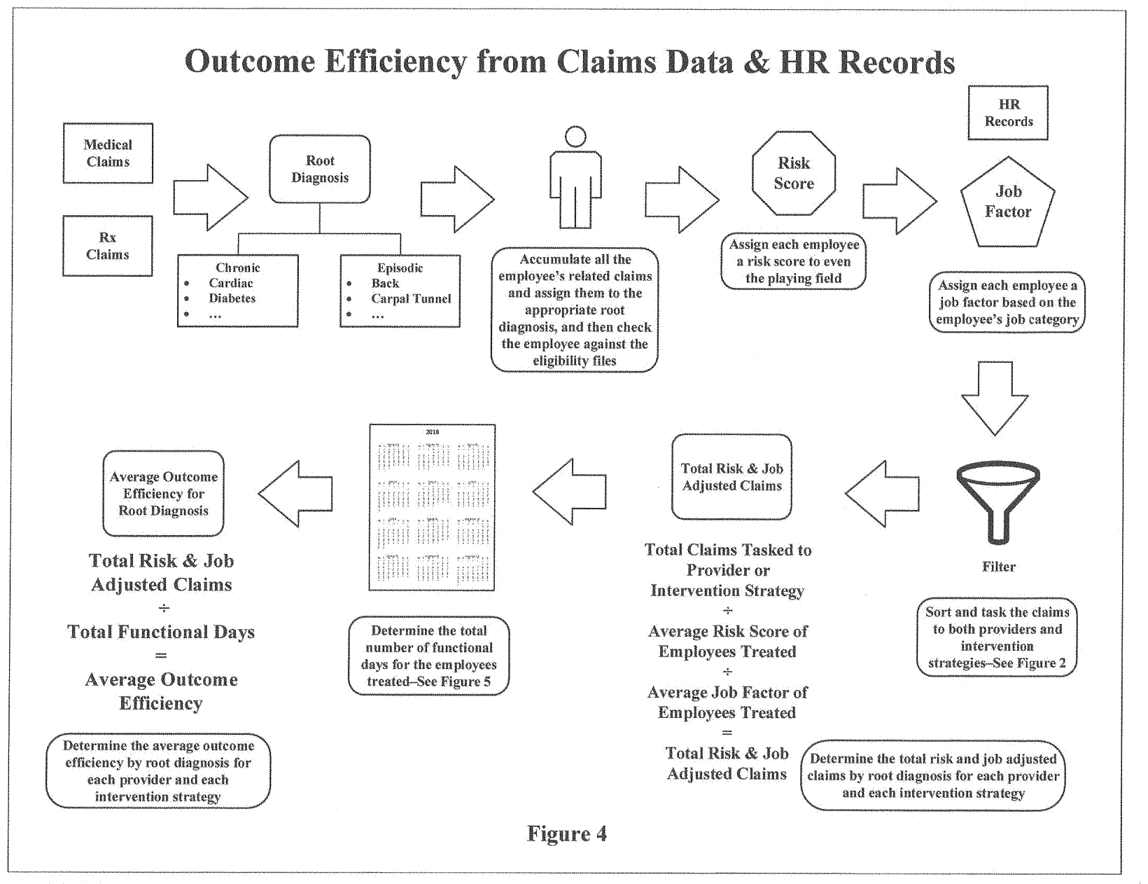

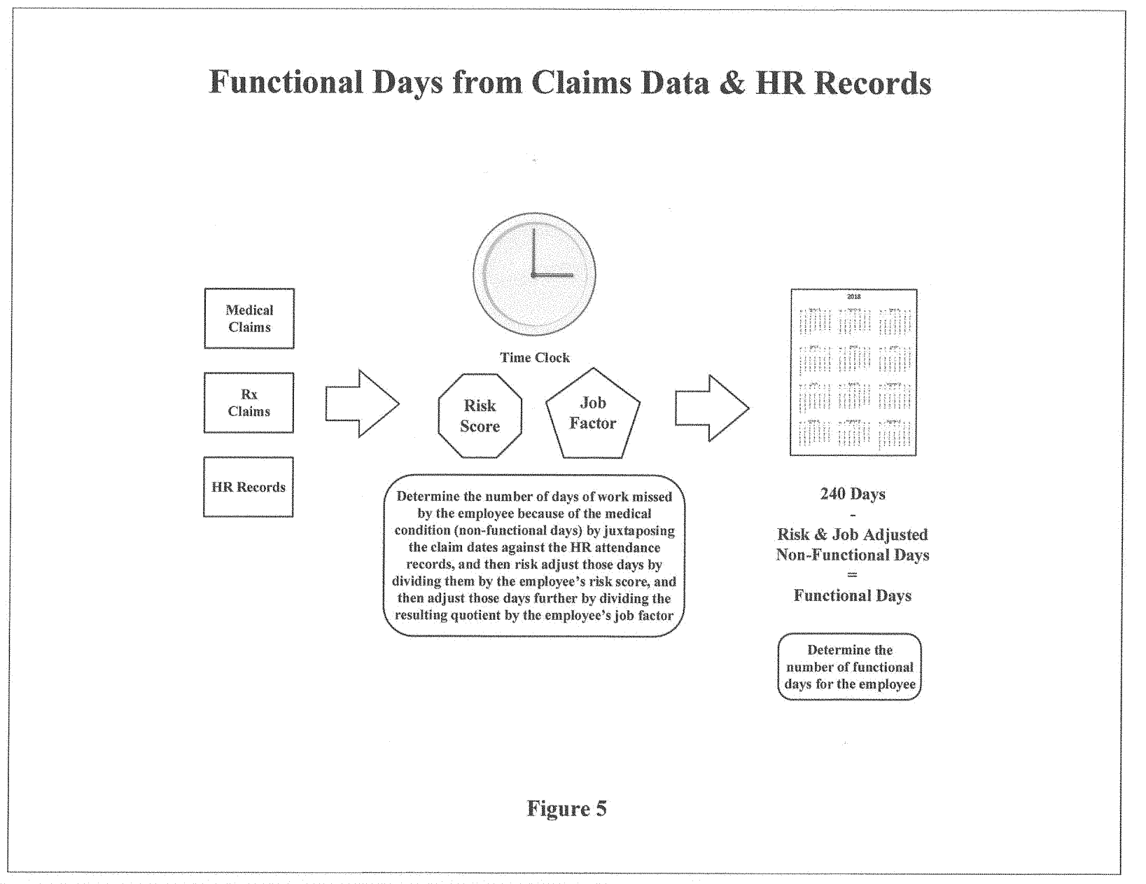

FIG. 4 depicts the computer search engine's flow when searching medical and pharmacy claims in combination with employer human resource records, with a cut-out for sorting and tasking the claims by both provider and intervention strategy, which is shown in FIG. 2, and a cut-out for determining an employee's functional days, which is shown in FIG. 5. As before, the search engine first organizes both the claims and the human resource records in tables with various headers that enable filtering, grouping and matching.

With the following exceptions, the flow when using claims and human resource records to determine the outcome efficiencies with respect to employees is the same as when using only claims data. The perspective of what constitutes a good outcome, however, now shifts from the patient (or in this case, the employee) to that of the employer; and a good outcome for the employer is having the employee at work.

Total possible functional days from an employer's perspective are the number of work days in the employer's year. For a typical employer, the number of work days in a year would be 240-five days per week (Monday through Friday) for the 52 weeks in a year, less a two-week (ten work day) vacation, less the ten national holidays recognized by the federal government ((5×52)−(2×5)−10=240). The search engine therefore uses 240 days as the default for the total possible functional days over a one-year measurement period.

The search engine juxtaposes the dates of the claims for an employee's root diagnosis against the human resource attendance records and determines the days that the employee missed work due to that condition (e.g. days missed within a designated period before or after a claim are considered missed because of the condition)—these are the employee's non-functional days. As before, the number of non-functional days is risk-adjusted by dividing by the employee's risk score.

An additional feature that can be deployed by the search engine when using employer human resource records (but which is not required to be deployed), is to create a numerical job factor for each employee based on that employee's job. Elements that weigh on that factor include the physical exertion that the job requires, time spent standing versus sitting, repetitive stress movements, and the emotional, mental and physical stress of the job. The job factor can be deployed in the determination of the outcome efficiency of a provider or intervention strategy with respect to a specific employee with a particular root diagnosis—and the average outcome efficiency for that root diagnosis for a provider or intervention strategy—much like the risk score.

Functional days are then the total number of work days in the measurement period, 240 when the measurement period is a year, less the adjusted number of non-functional days.

Continuing the example from above, take the PCP that treated ten patients during the year for diabetes (and assume that all ten were employees) with a total claims costs tasked to the PCP of $19,500. The average risk score of the ten employees was 1.300, so the PCP's total risk-adjusted claims cost was $15,000 ($19,500=1.300=$15,000). Now assume that the average job factor for these ten employees was 1.100, which would make the total risk and job adjusted costs $13,636 ($15,000÷1.100=$13,636). If the ten employees had 280 non-functional days because of their diabetes (of the 300 non-functional days from the previous example, 20 of those days occurred on non-work days), the risk-adjusted number of non-functional days would be 215 (280÷1.300=215), and then the job factor adjusted number of non-functional days on top of that would be 195 (215÷1.100=195). The total functional days would therefore be 2,205 ((10×240)−195=2,205). Accordingly, the PCP's average outcome efficiency for treating these ten employees for diabetes would be $6.18 ($13,636÷2,205=$6.18). In other words, $6.18 is the average claims cost per day to keep these ten employees at work.

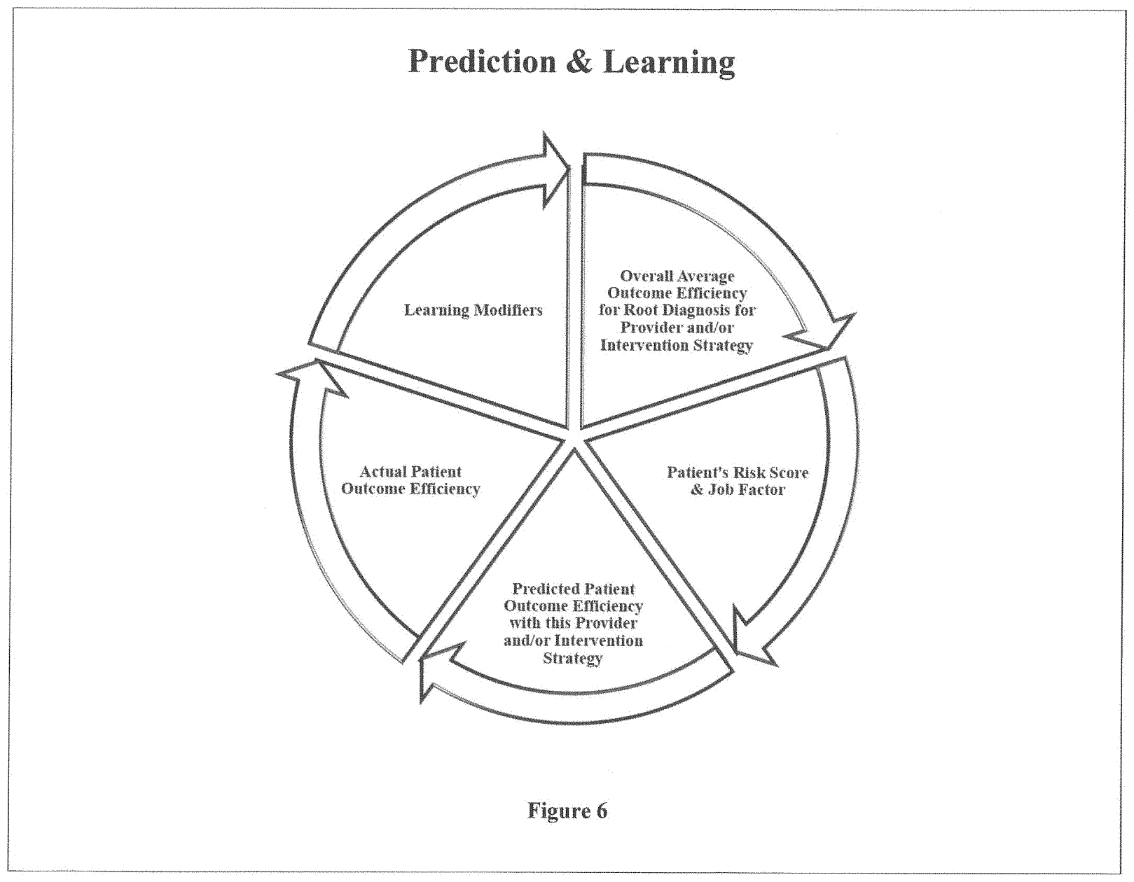

The invention now shifts from determining the overall average outcome efficiency of a provider or intervention strategy when treating a root diagnosis, to predicting the outcome efficiency with respect to a particular patient and/or employee when going to that provider for treatment of that condition or undergoing that intervention strategy, which is depicted in FIG. 6.

The prediction is determined by: (1) taking the overall average outcome efficiency for that root diagnosis of the provider or intervention strategy, (2) multiplying it by the risk score of the particular patient or employee (1.000 for scores of 1.000 and below, the actual score for scores above 1.000), and then if an employee, (3) multiplying that product by the employee's job factor (if the job factor feature has been deployed).

The prediction is then compared to the actual outcome efficiency achieved. This comparison can be performed [symbol] future data when the actual outcome efficiency is unknown, which would be the case when an individual is using the search engine to select a provider.

Regression analysis is then employed to modify the risk score and job factor as they effect the root diagnosis, with the modifying factors deployed as additional elements in the prediction formula. This analysis may address components or subsets of the risk score and job factor, such as whether a diabetic condition (a factor in the risk score) should be given more or less weight when predicting the total costs for a particular root diagnosis.

The revised prediction is then compared to the actual outcome efficiency achieved, and the modifying factors adjusted in a “loop” of neural network learning until the predicted outcome efficiency equals the actual outcome efficiency. Accordingly, the invention is a self-teaching outcome-based artificial intelligence search engine.

This detailed description is not intended to limit or represent an exhaustive enumeration of the principles disclosed. It will be apparent to those of skill in the art that numerous changes may be made in such details without departing from the spirit of the disclosed principles, and that the invention does not require all the features described above to be deployed for the invention to function.

We claim:

1. A method of searching claims in a computing environment for the healthcare providers and/or intervention strategies with the best outcome efficiencies when treating a particular root diagnosis, comprising:
organizing medical and/or pharmacy claims in tables, including pooling claims from different sources;
identifying the root diagnoses for each patient's claims;
accumulating all the patient's claims attributable to a root diagnosis over the entire continuum of care, and then grouping those claims by the specified measurement period, with the default measurement period being an annual period;
organizing the eligibility files for any applicable health plan or other program in tables, and then determining whether each patient participated in that plan or program for the entirety of each measurement period;
determining a risk score for each patient using the age, gender, diagnoses, and in some cases, drug prescriptions data contained in the claims;
organizing the healthcare providers and intervention strategies in tables;
tasking each provider that filed a claim grouped with a patient's root diagnosis with both: (1) that provider's claims grouped with that root diagnosis, and (2) all “downstream” claims from direct and indirect referrals of the patient made by that provider to other providers;
sorting the providers into categories, such as: (1) primary care physicians (PCPs), (2) non-surgeon specialists, (3) surgeons, and (4) institutions, such as hospitals and out-patient centers;
similarly sorting the claims by the intervention strategies for each root diagnosis and tasking each such strategy with both the direct claims of that intervention strategy and the indirect “downstream” claims stemming from it;
determining per measurement period the risk-adjusted claims of each provider and/or intervention strategy to treat a patient with a particular root diagnosis by: (1) combining all the patient's claims tasked to that provider and/or intervention strategy when treating that root diagnosis (including downstream costs), and (2) dividing those total costs by the patient's risk score;
determining per measurement period the total risk-adjusted claims of each provider and/or intervention strategy to treat patients with a particular root diagnosis by: (1) combining all the claims tasked to that provider and/or intervention strategy when treating that root diagnosis (including downstream costs), and (2) dividing those total costs by the average risk score of the patients with that root diagnosis that the provider treated or who underwent that intervention strategy;
identifying from the claims the non-functional days of each patient attributable to the root diagnosis during the measurement period;
risk-adjusting the non-functional days by dividing them by the patient's risk score;
determining the functional days for the patient by subtracting the adjusted non-functional days from the number of days in the measurement period;
determining per measurement period the outcome efficiency for a provider and/or intervention strategy when treating a particular patient with a root diagnosis by taking the adjusted claims of that patient tasked to that provider and/or intervention strategy and dividing by the patient's number of functional days, which results in the adjusted claims cost per day to keep that patient functional;
determining per measurement period the average outcome efficiency for a provider and/or intervention strategy when treating a root diagnosis by taking the total adjusted claims tasked to that provider and/or intervention strategy when treating that diagnosis and dividing by the total functional days of all the patients that the provider treated for it or who underwent that intervention strategy;
ranking the providers by category and the intervention strategies for each root diagnosis based on their average outcome efficiencies over all relevant measurement periods—from the best with the lowest outcome efficiency, to the worst with the highest;
directing a person through the search engine to the providers and/or intervention strategies with the best outcome efficiencies for that person's particular problem (i.e. root diagnosis);
filtering the results displayed via drop-down menus and/or similar techniques by variables, such as: (1) root diagnosis, (2) provider category, (3) geographic proximity, and (4) provider network (in-network versus in or out of network); and
filtering the results displayed based on the type of user: (1) for a plan, provider network, employer or other administrative user, the search engine displays the overall average outcome efficiency by root diagnosis of each healthcare provider treating patients with that diagnosis, and each available intervention strategy for that diagnosis, including configurations and subsets in various dashboards and reports, (2) for PCPs and other providers using the search engine to make patient referrals or choose from several intervention strategies, the search engine displays: (A) a list of the surgeons, specialists and institutions with overall average outcome efficiencies better than a designated threshold, and/or (B) the possible intervention strategies and their overall average outcome efficiencies, and (3) for patients and other individuals seeking treatment the search engine displays all the providers, including PCPs, with overall average outcome efficiencies better than a designated threshold (and may, or may not, display the overall average outcome efficiencies for the intervention strategies).

2. The method of claim 1, as well as comprising:
determining from the claims data the risk score of the patient or other individual using the search engine; and
predicting the outcome efficiency of each provider and/or intervention strategy when treating that patient or other individual for a root diagnosis by multiplying the risk score of that patient or individual by the overall average outcome efficiency for that root diagnosis of the provider and/or intervention strategy.

3. The method of claim 2, as well as comprising:
comparing the predicted outcome efficiency for the patient or other individual with the actual outcome efficiency achieved;
employing regression analysis to modify the risk score (and/or components or subsets thereof) as they affect the root diagnosis, with the modifying factors deployed as additional elements in the prediction formula; and
comparing the revised prediction to the actual outcome efficiency, and then adjusting the modifying factors in a “loop” of neural network learning until the predicted outcome efficiency equals the actual outcome efficiency.

4. A method of searching claims and human resource records in a computing environment for the healthcare providers and/or intervention strategies with the best outcome efficiencies when treating a particular root diagnosis, comprising:
organizing the medical and/or pharmacy claims under an employer's health plan and/or workers' compensation program in tables, including pooling the claims from different employers;
organizing the employer human resource records (e.g. employee absence and job descriptions) in tables, including pooling the human resource records from different employers;
identifying the root diagnoses for each employee's claims;
accumulating all the employee's claims attributable to a root diagnosis over the entire continuum of care, and then grouping those claims by the specified measurement period, with the default measurement period being an annual period;
organizing the eligibility files for any applicable health plan or other program in tables, and then determining whether each employee participated in that plan or program for the entirety of each measurement period;
determining a risk score for each employee using the age, gender, diagnoses, and in some cases, drug prescriptions data contained in the claims and/or human resource records;
creating a numerical job factor for each employee based on the information contained in the human resource records;
organizing the healthcare providers and intervention strategies in tables;
tasking each provider that filed a claim grouped with an employee's root, diagnosis with both: (1) that provider's claims grouped with that root diagnosis, and (2) all “downstream” claims from direct and indirect referrals of the employee made by that provider to other providers;
sorting the providers into categories, such as: (1) PCPs, (2) non-surgeon specialists, (3) surgeons, and (4) institutions, such as hospitals and out-patient centers;
similarly sorting the claims by the intervention strategies for each root diagnosis and tasking each such strategy with both the direct claims of that intervention strategy and the indirect “downstream” claims stemming from it;
determining per measurement period the risk and job adjusted claims of each provider and/or intervention strategy to treat an employee with a particular root diagnosis by: (1) combining all the employee's claims tasked to that provider and/or intervention strategy when treating that root diagnosis (including downstream costs), (2) dividing those total costs by the employee's risk score, and (3) dividing that resulting quotient by the employee's job factor;
determining per measurement period the total risk and job adjusted claims of each provider and/or intervention strategy to treat employees with a particular root diagnosis by: (1) combining all the claims tasked to that provider and/or intervention strategy when treating that root diagnosis (including downstream costs), (2) dividing those total costs by the average risk score of the employees with that root diagnosis that the provider treated or who underwent that intervention strategy, and (3) dividing that resulting quotient by the average employee job factor of those employees;
identifying from the claims and human resource records the days missed from work (i.e. non-functional days) of each employee attributable to the root diagnosis during the measurement period;
risk-adjusting the non-functional days by dividing them by the employee's risk score, and then further dividing that quotient by the employee's job factor;
determining the functional days for the employee by subtracting the adjusted non-functional days from the number of work days in the measurement period;
determining per measurement period the outcome efficiency for a provider and/or intervention strategy when treating a particular employee with a root diagnosis by taking the adjusted claims of that employee tasked to that provider and/or intervention strategy and dividing by the employee's number of functional days, which results in the adjusted claims cost per day to keep that employee at work (i.e. functional);
determining per measurement period the average outcome efficiency fora provider and/or intervention strategy when treating a root diagnosis by taking the total adjusted claims tasked to that provider and/or intervention strategy when treating that diagnosis and dividing by the total functional days of all the employees that the provider treated for it or who underwent that intervention strategy;
ranking the providers by category and the intervention strategies for each root diagnosis based on their average outcome efficiencies over all relevant measurement periods—from the best with the lowest outcome efficiency, to the worst with the highest;
directing a person (whether an employee or not) through the search engine to the providers and/or intervention strategies with the best outcome efficiencies for that person's particular problem (i.e. root diagnosis);
filtering the results displayed via drop-down menus and/or similar techniques by variables, such as: (1) root diagnosis, (2) provider category, (3) geographic proximity, and (4) provider network (in-network versus in or out of network); and
filtering the results displayed based on the type of user: (1) for a plan, provider network, employer or other administrative user, the search engine displays the overall average outcome efficiency by root diagnosis of each healthcare provider treating employees with that diagnosis, and each available intervention strategy for that diagnosis, including configurations and subsets in various dashboards and reports, (2) for PCPs and other providers using the search engine to make patient referrals or choose from several intervention strategies, the search engine displays: (A) a list of the surgeons, specialists and institutions with overall average outcome efficiencies better than a designated threshold, and/or (B) the possible intervention strategies and their overall average outcome efficiencies, and (3) for individuals (whether employees or not) the search engine displays all the providers, including PCPs, with overall average outcome efficiencies better than a designated threshold (and may, or may not, display the overall average outcome efficiencies for the intervention strategies).

5. The method of claim 4, as well as comprising:
determining from the claims and/or human resource records the risk score of the employee or other individual using the search engine;
determining from the human resource records the job factor of that person if he or she is an employee; and
predicting the outcome efficiency of each provider and/or intervention strategy when treating that employee or other individual for a root diagnosis by multiplying the risk score of that employee or individual by the overall average outcome efficiency for that root diagnosis of the provider and/or intervention strategy, and then multiplying that product by that person's job factor if he or she is an employee.

6. The method of claim 5, as well as comprising:
comparing the predicted outcome efficiency for the employee or other individual with the actual outcome efficiency achieved;
employing regression analysis to modify the risk score and job factor (and/or components or subsets thereof) as they affect the root diagnosis, with the modifying factors deployed as additional elements in the prediction formula; and
comparing the revised prediction to the actual outcome efficiency, and then adjusting the modifying factors in a “loop” of neural network learning until the predicted outcome efficiency equals the actual outcome efficiency.
